(19) United States
(12) Patent Application Publication
(10) Pub. No.: US 2010/0050472 A1
(43) Pub. Date: Mar. 4, 2010

(54) STABILIZING INSOLE AND METHOD FOR USING THE STABILIZING INSOLE

(76) Inventors: Tzann-Yuh TZENG, Taipei (TW); Min-Te LIN, Taipei (TW)

Correspondence Address: Hershkovitz & Associates, LLC, 2845 Duke Street, Alexandria, VA, 22314, US

(21) Appl. No.: 12/203,628

(22) Filed: Sep. 3, 2008

(51) Int. Cl. A43B 7/16; A43B 7/14; A43B 13/38; A43B 19/00; A61F 5/14

(52) U.S. Cl. 36/92; 36/88; 36/43; 36/71; 36/141

(57) ABSTRACT

A stabilizing insole has a base, a heel pad and a forefoot pad. The heel pad is formed on the base and has a heel supporting segment. The forefoot pad is formed on the base and has a forefoot supporting section. The heel and forefoot pads concentrates force applied by a person's foot at natural balance points so the user does not twist his or her ankle.

### BACKGROUND OF THE INVENTION

1. Field of the Invention

The present invention relates to an insole, and especially to a stabilizing insole that can be mounted in one or more positions in a shoe to provide increased stability for a person wearing the shoe so the person is less likely to lose their balance, fall or turn their ankle.

2. Description of Related Art

With the rise and increased interest in human engineering in society, shoes are a subject of human engineering as seen by air cushions in conventional soft-shoes and shapes of upper surfaces of soles to conform to people's feet. However, legs of almost every person are at least a slightly different length. Even slight differences in leg length can cause a person's joints to hurt especially in modern soft-shoes and conformal shoes. Often, a doctor attempts to correct bad posture with corrective shoes. However, the effect of wearing corrective shoes in correcting posture may not be obvious, and the price of corrective shoes is high.

To overcome the shortcomings, the present invention provides a stabilizing insole and a method for using the stabilizing insole to mitigate or obviate the aforementioned problems.

### SUMMARY OF THE INVENTION

The main objective of the invention is to provide a stabilizing insole that can be mounted in one or more positions in a shoe to provide increased stability for a person wearing the shoe so the person is less likely to lose their balance, fall or turn their ankle.

A stabilizing insole in accordance with the present invention comprises a base, a heel pad and a forefoot pad. The heel pad is formed on the base and has a heel supporting segment. The forefoot pad is formed on the base and has a forefoot supporting section. The heel and forefoot pads concentrates force applied by a person's foot at natural balance points so the user does not twist his or her ankle.

Other objectives, advantages and novel features of the invention will become more apparent from the following detailed description when taken in conjunction with the accompanying drawings.

### BRIEF DESCRIPTION OF THE DRAWINGS

FIG. 1 is a top view of a first embodiment of a heel pad of a stabilizing insole in accordance with the present invention;

FIG. 2 is a cross sectional side view of one variant of the heel pad of the stabilizing insole in FIG. 1;

FIG. 3 is a cross sectional view of another variant of the heel pad of the stabilizing insole in FIG. 1;

FIG. 4 is a top view of the forefoot pad of the stabilizing insole in FIG. 1;

FIG. 5 is an top view of the stabilizing insole in FIG. 1 including the base, the heel pad and the forefoot pad;

FIG. 6 is an operational cross sectional side view of the stabilizing insole in FIG. 5 with a foot stepping on the stabilizing insole;

FIG. 7 is a perspective view of a slipper integrated with the stabilizing pads in FIG. 5; and

FIG. 8 is an operational cross sectional side view of a second embodiment of a stabilizing insole in accordance with the present invention.

### DETAILED DESCRIPTION OF THE PREFERRED EMBODIMENT

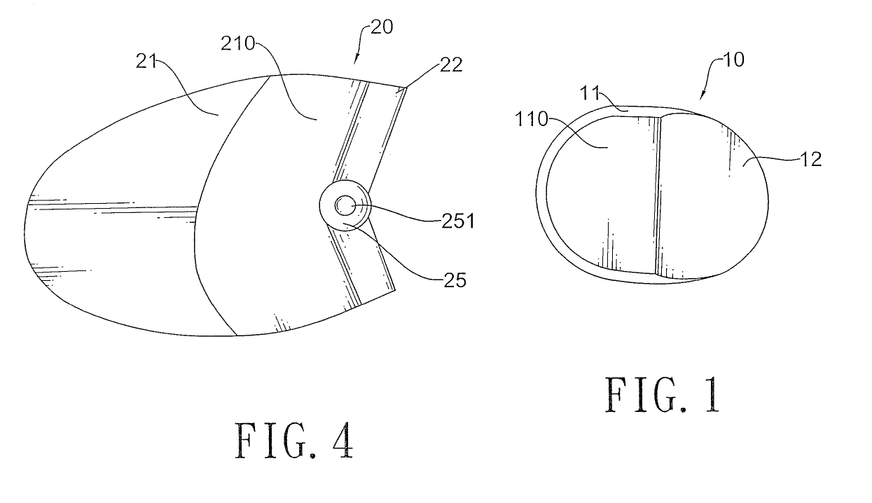

With reference to FIGS. 1 to 6, a first embodiment of a stabilizing insole in accordance with the present invention comprises a base (30), a heel pad (10, 10a) and a forefoot pad (20) and may further have a massaging bulge (25).

The base (30) is flat, is shaped to correspond to a shoe sole and has a top surface and a bottom surface. The top and bottom surfaces are flat and parallel to each other.

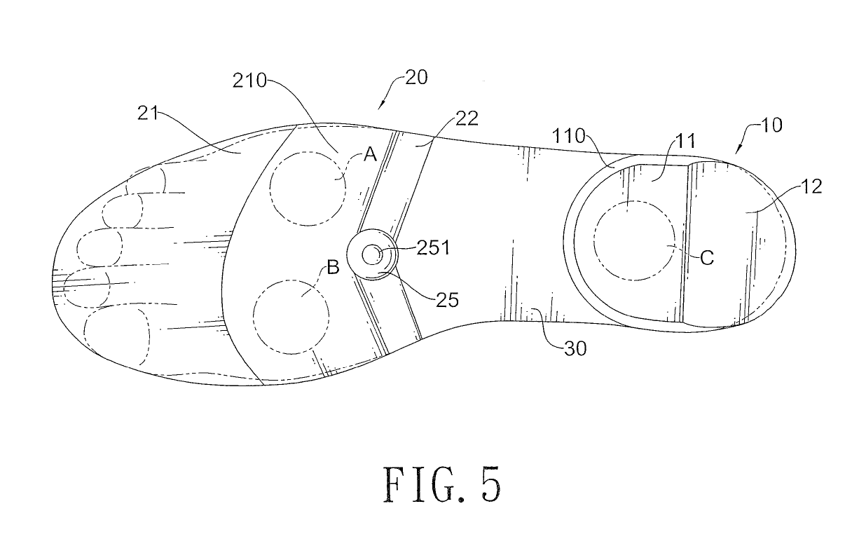

The heel pad (10, 10a) may be oval, is formed on the base (30) and protrudes from the top surface of the base (30). The heel pad (10, 10a) contacts and supports a heel of a user's foot in a contacting area (C), as shown in FIG. 5. The heel pad (10, 10a) has an outer surface and a heel supporting segment (110) and may further have an inclined front surface (11) and a sloping rear surface (12).

The outer surface is a top surface in the first embodiment.

The heel supporting segment (110) is formed on the heel pad (10, 10a), mainly supports the heel of the foot and has a flat outer surface parallel to the top and bottom surfaces of the base (30).

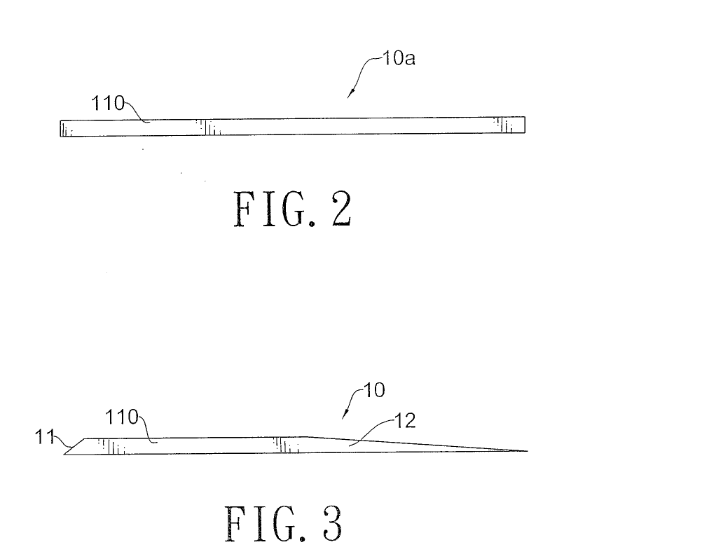

In a variant of the heel pad (10a), the heel supporting segment (110) is formed entirely on the heel pad (10a) so that the outer surface of the heel pad (10a) in uniformly flat, as shown in FIG. 2.

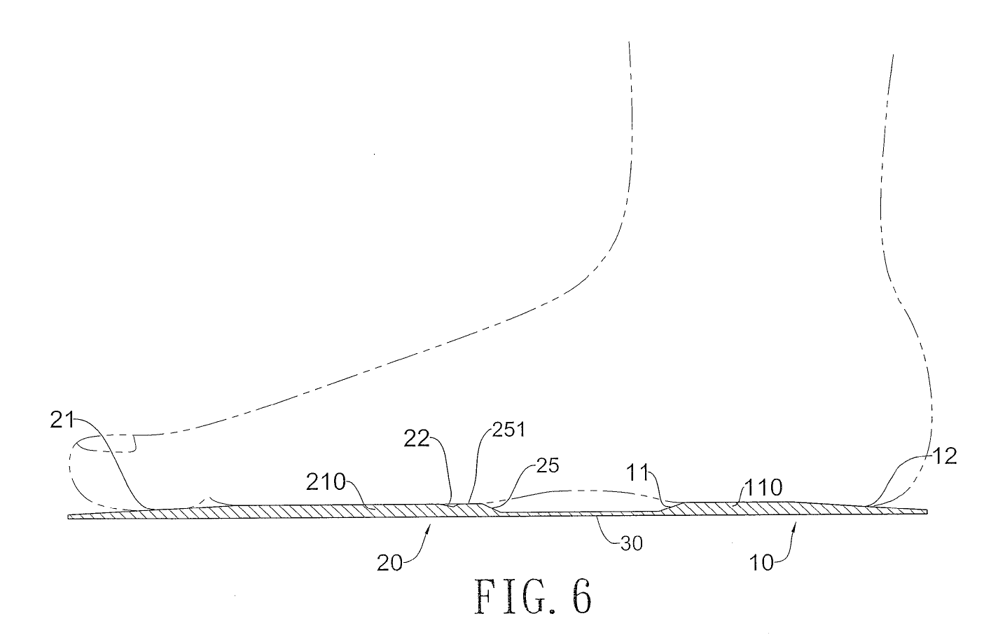

In another variant of the heel pad (10), the flat outer surface of the heel supporting segment (110) is substantially semi-oval and has a curved front edge and a linear rear edge. The inclined front surface (11) is U-shaped and is formed on outer surface of the heel pad (10) adjacent to the curved front edge of the heel supporting segment (110). A thickness of the heel pad (10) corresponding to the inclined front surface (11) is gradually decreased from the curved front edge. The sloping rear surface (12) is substantially semi-oval and is formed on the outer surface of the heel pad (10) adjacent to the linear rear edge of the heel supporting segment (110), as shown in FIGS. 5 and 6. A thickness of the heel pad (10) corresponding to the sloping rear surface (12) is gradually decreased from the linear rear edge. Furthermore, the heel supporting segment (110) has rigidity higher than rigidity of parts of the heel pad (10) corresponding respectively to the inclined front surface (11) and the sloping rear surface (12).

The forefoot pad (20) is tongue shaped, corresponds uniquely to a right or left foot, is attached to the base (30) and is formed on the base (30) and protrudes from the top surface of the base (30). The forefoot pad (20) contacts the heel of the foot in two transversely arranged contacting areas (A, B), as shown in FIG. 5. The forefoot pad (20) has an outer surface and a forefoot supporting section (210) and may further have an inclined front surface (21) and a concave rear surface (22).

The outer surface of the forefoot pad (20) in the first embodiment is a top surface.

The forefoot supporting section (210) is formed on the forefoot pad (20) and has a flat upper surface parallel to the top and bottom surfaces of the base (30). The forefoot supporting section (210) mainly supports a forefoot of the foot.

In a variant of the forefoot pad (20), the forefoot supporting section (210) has a convex front edge and a concave rear edge. The inclined front surface (21) is formed on the outer surface of the forefoot pad (20) adjacent to the convex front edge of the forefoot supporting section (210). A thickness of the forefoot pad (20) corresponding to the inclined front surface (21) is gradually decreased from the convex front edge. The concave rear surface (22) is formed on the outer surface of the forefoot pad (20) adjacent to the concave rear edge of the forefoot supporting section (210). A thickness of the forefoot pad (20) corresponding to the concave rear surface (22) is gradually decreased from the concave rear edge. Furthermore, the forefoot supporting section (210) has rigidity higher than rigidity of parts of the forefoot pad (20) corresponding respectively to the inclined front surface (21) and the concave rear surface (22).

The massaging bulge (25) is formed on the top base (30), protrudes from the top surface of the base (30), is located adjacent to the forefoot pad (20) and may press and massage a Chinese pressure point named “yongquan” on the sole of the foot to stimulate and maintain health of the kidneys. Yongquan is located between the first and second metatarsals of the foot near the arch. The massaging bulge (25) has a tip (251) that may be flat and presses the yongquan. The massaging bulge (25) may be formed adjacent the concave rear surface (22).

A method for using the stabilizing insole in accordance with the present invention comprises a providing step and a mounting step.

The providing step comprises providing a stabilizing insole as described above.

The mounting step comprises mounting the stabilizing insole with the heel supporting segment (110, 110b) of the heel pad (10, 10a, 10b) and the forefoot supporting section (210, 210b) of the forefoot pad (20, 20b) in a shoe to support a user's heel and forefoot of a foot.

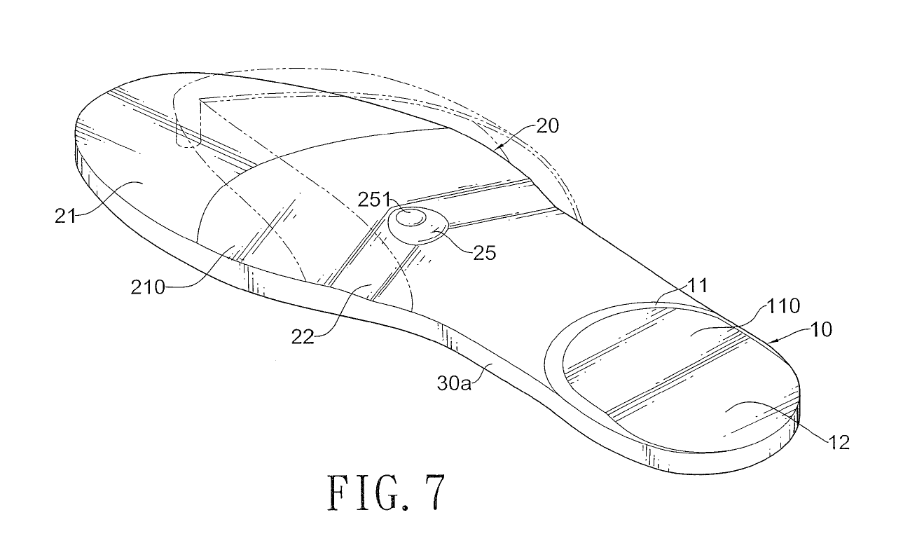

With further reference to FIG. 7, the stabilizing insole may be formed on one of a pair of slippers and positioned to correspond to a user's heel or forefoot. The base (30a) serves as a sole of the slipper.

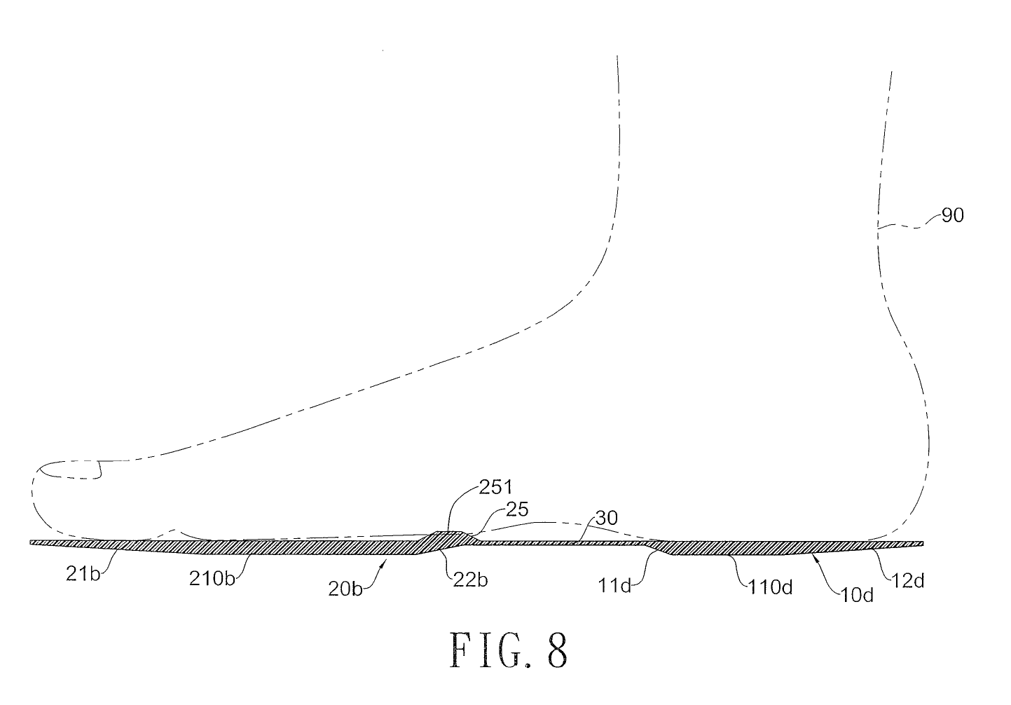

With reference to FIG. 8, a second embodiment of a stabilizing insole is similar to the first embodiment however the heel pad (10b) is formed on and protrudes from the bottom surface of the base (30) and the outer surface of the heel pad (10b) is a bottom surface. The forefoot pad (20b) is formed on and protrudes from the bottom surface of the base (30) and the outer surface of the heel pad (20b) is a bottom surface. The massaging bulge (25) is formed on and protrudes from the top surface of the base (30).

The stabilizing insole supports a user's feet to decrease the area of force and form three support points so the user does not twist his or her ankle.

Even though numerous characteristics and advantages of the present invention have been set forth in the foregoing description, together with details of the structure and function of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. A stabilizing insole comprising:
a base being flat and having a top surface and a bottom surface, and the top and bottom surfaces being flat and parallel to each other;
a heel pad formed on the base and having
an outer surface; and
a heel supporting segment formed on the heel pad and having a flat outer surface being parallel to the top and bottom surfaces of the base;
a forefoot pad tongue shaped, formed on the base and having
an outer surface; and
a forefoot supporting section formed on the forefoot pad and having a flat upper surface parallel to the top and bottom surfaces of the base.

2. The stabilizing insole as claimed in claim 1, wherein the heel supporting segment of the heel pad is formed entirely on the heel pad.

3. The stabilizing insole as claimed in claim 1, wherein:
the flat outer surface of the heel supporting segment of the heel pad has a curved front edge and a linear rear edge;
the heel pad further has
an inclined front surface being U-shaped and formed on outer surface of the heel pad adjacent to the curved front edge of the heel supporting segment, wherein a thickness of the heel pad corresponding to the inclined front surface is gradually decreased from the curved front edge; and
a sloping rear surface formed on the outer surface of the heel pad adjacent to the linear rear edge of the heel supporting segment, wherein a thickness of the heel pad corresponding to the sloping rear surface is gradually decreased from the linear rear edge.

4. The stabilizing insole as claimed in claim 3, wherein the heel supporting segment has rigidity higher than rigidity of parts of heel pad corresponding respectively to inclined front surface and the sloping rear surface.

5. The stabilizing insole as claimed in claim 4, wherein the flat outer surface of the heel supporting segment of the heel pad is substantially semi-oval.

6. The stabilizing insole as claimed in claim in claimed 5, wherein the sloping rear surface is substantially semi-oval.

7. The stabilizing insole as claimed in claim 1, wherein:
the forefoot supporting section of the forefoot pad has a convex front edge and a concave rear edge;
the forefoot pad further has
an inclined front surface formed on the outer surface of the forefoot pad adjacent to the convex front edge of the forefoot supporting section, wherein a thickness of the forefoot pad corresponding to the inclined front surface is gradually decreased from the convex front edge; and
a concave rear surface formed on the outer surface of the forefoot pad adjacent to the concave rear edge of the forefoot supporting section, wherein a thickness of the forefoot pad corresponding to the concave rear surface is gradually decreased from the concave rear edge.

8. The stabilizing insole as claimed in claim 7, where the forefoot supporting section of the forefoot pad has rigidity higher than rigidity of parts of the forefoot pad corresponding respectively to the inclined front surface and the concave rear surface.

9. The stabilizing insole as claimed in claim 1 further has a massaging bulge formed on the base located adjacent to the forefoot pad.

10. The stabilizing insole as claimed in claim 9, wherein the massaging bulge has a tip being flat.

11. The stabilizing insole as claimed in claim 10, wherein the heel pad, the forefoot pad and the massaging bulge protrude from the top surface of the base.

12. The stabilizing insole as claimed in claim 10, wherein the heel pad and the forefoot pad protrude from the bottom surface of the base, and the massaging bulge protrudes from the top surface of the base.

13. A method for using the stabilizing insole comprising
a providing step comprising a stabilizing insole having a base, a heel pad formed on the base and a forefoot formed on the base, wherein the heel pad having a heel supporting segment, and the forefoot pad has a forefoot supporting section; and
a mounting step comprising mounting the stabilizing insole in a shoe to support a user's heel and forefoot of a foot.

14. The method as claimed in claim 13, wherein the stabilizing insole formed on one of a pair of slippers and the base of the stabilizing insole serves as a sole of the slipper.
